(19) United States
(12) Patent Application Publication
(10) Pub. No.: US 2025/0144085 A1
(43) Pub. Date: May 8, 2025

(54) PHARMACEUTICAL COMPOSITION FOR PREVENTING OR TREATING CANCER COMPRISING OMEPRAZOLE BROWNING MATTER

(71) Applicant: NEW CANCER CURE BIO CO., LTD, Gyeonggi-do (KR)

(72) Inventor: Soo Youl KIM, Goyang-si (KR)

(73) Assignee: NEW CANCER CURE-BIO CO., LTD., Goyang-si, Gyeonggi-do (KR)

(21) Appl. No.: 18/694,723

(22) PCT Filed: Sep. 23, 2022
(86) PCT No.: PCT/KR2022/014281

§ 371 Date: Mar. 22, 2024

(30) Foreign Application Priority Data
Sep. 23, 2021 (KR) 10-2021-0125559
Sep. 23, 2022 (KR) 10-2022-01202694

(51) Int. Cl. A61K 31/4439; A61K 45/06; A61P 35/00

(52) U.S. Cl. CPC A61K 31/4439; A61K 45/06; A61P 35/00

(57) ABSTRACT

The present invention relates to a pharmaceutical composition for the prevention or treatment of cancer, and anticancer adjuvants, comprising omeprazole (KN510) browning substance.

In the present invention, omeprazole browning substance (acid degradation substance) was prepared by treating conventional omeprazole with acid, and when the prepared omeprazole browning substance (acid degradation substance) was treated on a pancreatic cancer cell line, etc, it was confirmed that the anticancer effect was significantly increased compared to conventional omeprazole. Therefore, the omeprazole (KN510) browning substance (acid degradation substance) of the present invention can be utilized as a composition for preventing or treating cancer, or as an anticancer adjuvant.

### TECHNICAL FIELD

The present invention relates to a pharmaceutical composition and/or an anticancer adjuvant for preventing or treating cancer comprising omeprazole (KN510) browning matter.

### BACKGROUND ART

While normal cells are capable of regular, resilient proliferation and suppression as needed, cancer cells are capable of unrestricted proliferation, resulting in a cell mass of undifferentiated cells, also known as a tumor. These cancerous cells are a mass of undifferentiated cells, also known as a tumor, that proliferate uncontrollably, while the surrounding normal cells are capable of regular, resilient growth and suppression as needed. These cancer cells infiltrate the surrounding tissues and metastasize to other organs of the body, causing severe suffering and eventually death. Despite the advancement of medical science, the number of cancer patients in Korea continues to increase, increasing by about 44% in the last 10 years, and the international market for anticancer drugs has also increased, with a reported value of about $100 billion per year.

Chemotherapy for cancer treatment does not selectively target cancer cells, leading to many side effects. To solve this problem, proton-pump inhibitors (PPIs) have recently been used to block hydrogen transport out of tumor cells, thereby altering the acidity of the tumor microenvironment. This pH change promotes tumor cell death, reduces drug resistance, and reduces cancer metastasis.

Omeprazole, one of the proton pump inhibitors, is a commonly prescribed drug for the treatment of dyspepsia, peptic ulcer disease, erosive esophagitis, or eosinophilic esophagitis. However, omeprazole or its pharmaceutically acceptable salts are easily degraded and browning by acidic media such as stomach acid, so to prevent drug degradation by gastric juice, omeprazole is commercially available in forms that minimize drug degradation by gastric juice using formulation techniques such as enteric coating or enteric pellets (Korean Laid-open Patent No. 1999-0044631; Korean Registered Patent No. 10-2276547).

A recent study showed that proton pump inhibitors, including omeprazole, inhibit the activity of Fatty Acid Synthase (FASN), which helps cancer cells produce fatty acids that are key to their survival, and induce cancer cell death while minimizing the effects on non-cancerous cells (Walsh et al., Journal of Experimental & Clinical Cancer Research, 34:93, 2015).

### DISCLOSURE

### Technical Problem

Accordingly, as a result of our efforts to derive omeprazole derivatives with increased anticancer activity in this invention, we confirmed that omeprazole browning substances (acid degradation substances) in the pH range of 1^{˜}6 prepared by acid treatment to omeprazole have a significant increase in anticancer effect compared to conventional omeprazole, and completed the present invention.

Accordingly, it is an object of the present invention to provide a pharmaceutical composition for the prevention or treatment of cancer comprising omeprazole browning substance (acid degradation substance) or a pharmaceutically acceptable salt thereof as an active ingredient.

Another object of the present invention is to provide an anticancer adjuvant comprising omeprazole browning substance (acid degradation substance) or a pharmaceutically acceptable salt thereof as an active ingredient.

### Technical Solution

To fulfill the purposes described above,

The present invention provides a pharmaceutical composition for the prevention or treatment of cancer comprising omeprazole (KN510) browning substance or a pharmaceutically acceptable salt thereof as an active ingredient.

In addition, the present invention provides an anticancer adjuvant comprising omeprazole (KN510) browning substance (acid degradation substance) or a pharmaceutically acceptable salt thereof as an active ingredient.

According to a preferred embodiment of the present invention, the omeprazole (KN510) browning substance (acid degradation substance) may have a pH in the range of 1 to 6.

According to another preferred embodiment of the present invention, the omeprazole (KN510) browning substance (acid degradation substance) can be prepared by treating omeprazole (KN510) with an acid or acidic solution having a pH in the range of 1 to 6, wherein the acid or acidic solution can be 1 M hydrogen chloride (HCl), acetic acid, or lactic acid.

According to another preferred embodiment of the present invention, omeprazole (KN510) browning substance (acid degradation substance) can be prepared by treating 1 to 200 μl of 1 M hydrogen chloride (HCl), acetic acid or lactic acid per 100 μl of 100 mM omeprazole (KN510) and then reacting for 30 minutes to 2 hours at room temperature.

According to another preferred embodiment of the present invention, the cancer may be one or more cancers selected from the group consisting of colon cancer, lung cancer, gastric cancer, breast cancer, melanoma, prostate cancer, ovarian cancer, kidney cancer, pancreatic cancer, glioblastoma, and liver cancer.

According to another preferred embodiment of the present invention, the pharmaceutical composition or anticancer adjuvant may further comprise an additional anticancer agent, wherein the anticancer agent can be selected from the group consisting of one or more metabolic inhibitors selected from the group consisting of Irinotecan, fluorouracil, 5-FU), Paclitaxel, Gemcitabine, Cisplatin, Vermurafenib and pharmaceutically acceptable salts thereof; and/or one or more tumor immunosuppressants selected from the group consisting of Pembrolizumab, Nivolumab, Atezolizumab, Ipilimumab, and Durvalumab.

### Advantageous Effects

In the present invention, omeprazole browning substance (acid degradation substance) was prepared by treating conventional omeprazole (KN510) with acid, and when the prepared omeprazole browning substance (acid degradation substance) was treated to a pancreatic cancer cell line, a significant increase in anticancer effect was observed compared to conventional omeprazole. Therefore, the omeprazole (KN510) browning substance (acid degradation substance) of the present invention can be utilized as a composition for preventing or treating cancer, or as an anticancer adjuvant.

### DESCRIPTION OF DRAWINGS

FIG. 1 is a photograph of an observation of omeprazole acid or base degradation substances when omeprazole (KN510) is treated with an acid or base.

FIG. 2 is a photograph of omeprazole (KN510) treated with 1 M hydrogen chloride (HCl), showing omeprazole browning substance (acid degradation substance).

FIG. 3 is a photograph of omeprazole (KN510) treated with acetic acid, showing omeprazole browning substance (acid degradation substances).

FIG. 4 is a photograph of omeprazole (KN510) treated with lactic acid, showing omeprazole browning substance (acid degradation substance).

FIG. 5 shows the absorbance of omeprazole (KN510) when treated with 1 M hydrogen chloride (HCl).

FIG. 6 shows the absorbance of omeprazole (KN510) when treated with acetic acid.

FIG. 7 shows the absorbance data of omeprazole (KN510) when treated with lactic acid.

FIG. 8 shows data confirming the cancer cell killing effect of omeprazole (KN510) acid degradation substance or its basic degradation substance when treated on a pancreatic cancer cell line (MIA PaCa-2).

### MODE OF THE INVENTION

Hereinafter, the present invention will be described in more detail.

In one aspect, the present invention relates to a pharmaceutical composition for the prevention or treatment of cancer comprising omeprazole (KN510) browning substance or a pharmaceutically acceptable salt thereof as an active ingredient.

In another aspect, the present invention relates to an anticancer adjuvant comprising omeprazole (KN510) browning substance or a pharmaceutically acceptable salt thereof as an active ingredient.

In the present invention, the omeprazole (KN510) browning substance may be an acid degradation substance (also refer to “degradation substance by acid), which may be in the range of pH 1 to 6, preferably in the range of pH 2 to 3.

In the present invention, the omeprazole (KN510) browning substance (acid degradation substance) can be prepared by treating omeprazole (KN510) with an acid or an acidic solution in the range of pH 1 to 6, preferably in the range of pH 1 to 4. The acid can be 1 M hydrogen chloride (HCl), acetic acid or lactic acid.

In the present invention, the omeprazole (KN510) browning substance (acid degradation substance) can be prepared by treating 1 to 200 μ [symbol] of 1 M hydrogen chloride (HCl), acetic acid or lactic acid per 100 μ [symbol] of 100 mM omeprazole (KN510) and then reacting for 30 minutes to 2 hours at room temperature.

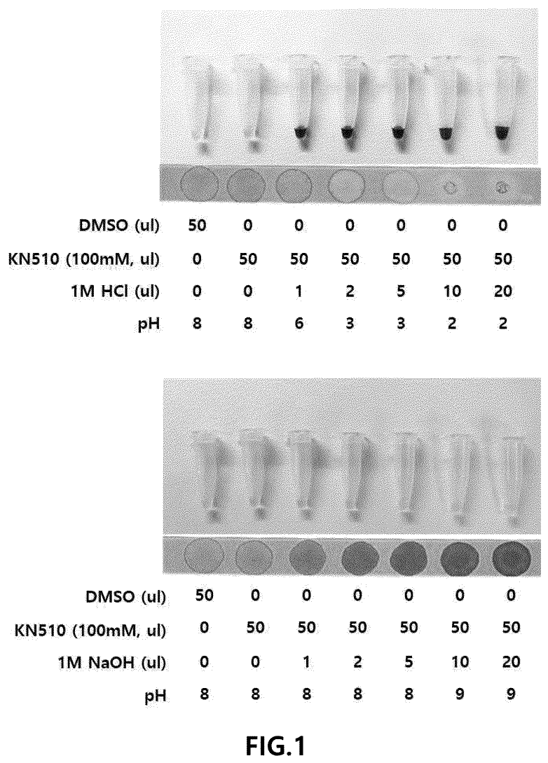

In a specific embodiment of the present invention, omeprazole (KN510) degradation substance was prepared by treating 50 μ [symbol] of 100 mM omeprazole (KN510) with 1 to 20 μ [symbol] of 1 M HCl or 1 M NaOH, respectively, and then reacting for 1 hour at room temperature, and observing the color and pH of the prepared degradation substance, as shown in FIG. 1, a browning phenomenon was observed for the omeprazole (KN510) acid degradation substance with a pH of 2 to 6.

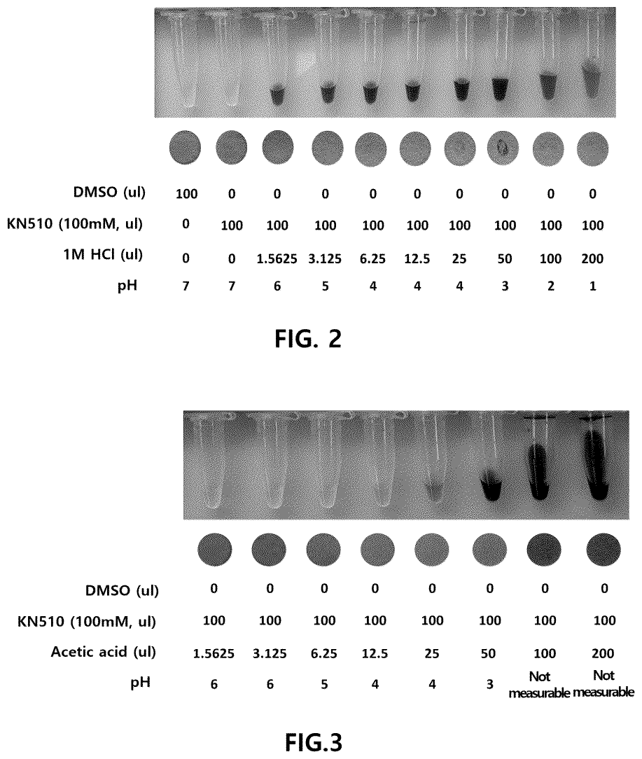

In addition, 100 μ [symbol] of 100 mM omeprazole (KN510) was added to 1 M HCl, acetic acid, and lactic acid at different concentrations (1.5625 μ [symbol] to 200 μ [symbol] ), respectively, and then reacted for 1 hour at room temperature to prepare omeprazole (KN510) acid degradation substance, and it was confirmed that omeprazole (KN510) browning substance (acid degradation substance) with pH 1^{˜}6 was prepared, as shown in FIGS. 2 to 4.

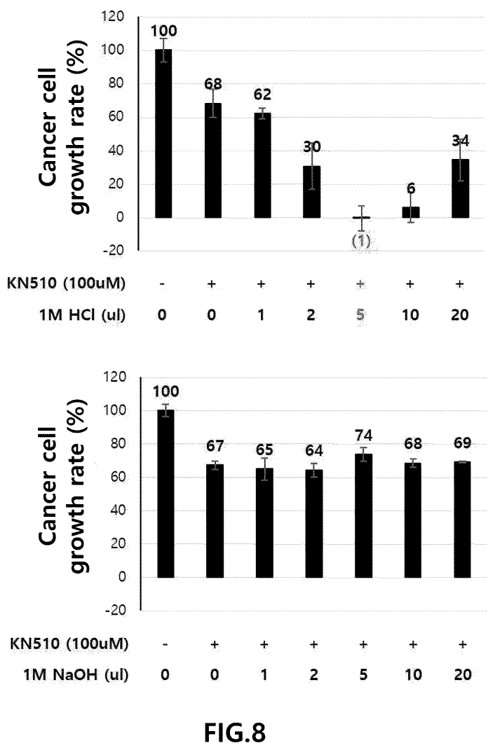

In another specific embodiment of the present invention, omeprazole (KN510) browning substance (acid degradation substance) and its base degradation substance prepared according to the present invention were treated to a pancreatic cancer cell line (MIA PaCa-2), respectively. As shown in FIG. 8, it was confirmed that omeprazole (KN510) browning substance (acid degradation substance) in the pH range of 1^{˜}6 showed higher anticancer activity compared to conventional omeprazole (KN510), and omeprazole (KN510) browning substance (acid degradation substance) in the pH range of 2^{˜}3 showed 60%^{˜}100% anticancer effect.

In particular, the anticancer effect of the present browning substance prepared by treating 50 μ [symbol] of 100 mM omeprazole (KN510) with 5 μ [symbol] of 1 M HCl was 100%, showing a superior anticancer effect compared to conventional omeprazole (KN510).

In the present invention, the cancer may be any one or more cancers selected from the group consisting of colon cancer, lung cancer, gastric cancer, breast cancer, melanoma, prostate cancer, ovarian cancer, kidney cancer, pancreatic cancer, glioblastoma, and liver cancer.

In the present invention, the composition or anticancer adjuvant may further comprise an additional anticancer agent, wherein the anticancer agent can be selected from the group consisting of one or more metabolic inhibitors selected from the group consisting of Irinotecan, fluorouracil, 5-FU), Paclitaxel, Gemcitabine, Cisplatin, Vermurafenib and pharmaceutically acceptable salts thereof; and/or one or more tumor immunosuppressants selected from the group consisting of Pembrolizumab, Nivolumab, Atezolizumab, Ipilimumab, and Durvalumab.

The composition of the present invention can be in a number of different formulations, either oral or parenteral. The composition may be formulated using one or more buffers (e.g., saline or PBS), antioxidants, bacteriostatic agents, chelating agents (e.g., EDTA or glutathione), fillers, bulking agents, binders, excipients (e.g., aluminum hydroxide), suspending agents, thickeners, wetting agents, disintegrating agents or surfactants, diluents or excipients.

Solid dosage forms for oral administration include tablets, disintegrating tablets, powders, granules, capsules, and the like, wherein one or more compounds are combined with at least one excipient, such as starch (including corn starch, wheat starch, rice starch, potato starch, and the like), calcium carbonate, sucrose, lactose, dextrose, sorbitol, mannitol, xylitol, erythritol maltitol, cellulose, methyl cellulose, sodium carboxymethylcellulose, and hydroxypropylmethyl-cellulose or gelatin. For example, the active ingredient may be compounded with solid excipients and then processed into a granular mixture after grinding and adding suitable auxiliaries to obtain a tablet or glycemic tablet.

In addition to simple excipients, lubricants such as magnesium stearate, talc, etc. are also used. Liquid formulations for oral administration include suspensions, solutions, emulsions, or syrups, which may contain a number of excipients, such as wetting agents, sweeteners, flavors, or preservatives, in addition to the commonly used simple diluents of water and liquid paraffin. In some cases, cross-linked polyvinylpyrrolidone, agar, alginate, or sodium alginate may be added as disintegrants, and further excipients may include anti-flocculants, lubricants, wetting agents, flavors, emulsifiers, and preservatives.

Formulations for parenteral administration include sterile aqueous solutions, non-aqueous solutions, suspensions, emulsions, lyophilized preparations, or suppositories. Non-aqueous solvents and suspensions may include propylene glycol, polyethylene glycol, vegetable oils such as olive oil, and injectable esters such as ethylolate. The base of the suppository can be witepsol, macrogol, tween 61, cacao paper, laurin paper, glycerol, gelatin, etc.

The composition of the present invention may be administered orally or parenterally and, when administered parenterally, may be formulated according to methods known in the art in the form of injectables for topical use; intraperitoneal, rectal, intravenous, intramuscular, subcutaneous, intrauterine dural, or cerebrovascular injection.

In the case of the injectables, they must be sterile and protected from contamination by microorganisms such as bacteria and fungi. Examples of suitable carriers for injectables include, but are not limited to, solvents or dispersion media comprising water, ethanol, polyols (e.g., glycerol, propylene glycol, and liquid polyethylene glycol), mixtures thereof, and/or vegetable oils. More preferably, suitable carriers include Hanks' solution, Ringer's solution, phosphate buffered saline (PBS) containing triethanolamine, or isotonic solutions such as sterile water for injection, 10% ethanol, 40% propylene glycol, and 5% dextrose. To protect the injectable from microbial contamination, the injectable may additionally contain various antibacterial and antifungal agents such as parabens, chlorobutanol, phenols, sorbic acid, thimerosal, and the like. In addition, the injectable may further comprise an isotonic agent such as sugar or sodium chloride in most cases.

The composition of the invention may be administered in a pharmaceutically effective amount. A pharmaceutically effective amount is an amount sufficient to treat a condition with a reasonable benefit/risk ratio applicable to medical treatment, and the effective dose level may be determined by factors including the type and severity of the patient's condition, the activity of the drug, sensitivity to the drug, time of administration, route of administration and elimination rate, duration of treatment, concomitant medications, and other factors well known in the medical field. The composition of the invention may be administered as individual therapeutic agents or in combination with other therapeutic agents, may be administered sequentially or concurrently with conventional therapeutic agents, and may be administered in single or multiple doses, i.e., the total effective amount of the composition of the invention may be administered to the patient in a single dose, or in a fractionated treatment protocol where multiple doses are administered over a longer period of time. Taking all of the above factors into consideration, it is important to administer an amount that achieves maximum effect in a minimal amount without side effects, which can be readily determined by those skilled in the art.

The preferred dosage of the composition depends on the patient's condition, body weight, severity of disease, drug form, route of administration, and duration, but may be suitably selected by those skilled in the art, for example from 0.0001 to 2,000 mg/kg per day, and more preferably from 0.001 to 2,000 mg/kg per day. The dose may be administered once a day, or may be divided into several doses. However, the above dosages do not limit the scope of the invention.

The composition of the present invention can be used alone or in combination with methods using surgery, radiation therapy, hormonal therapy, chemotherapy, and biological response modifiers.

The anticancer adjuvant of the present invention refers to any form intended to increase the anticancer effect of an anticancer drug or to suppress or improve the side effects of an anticancer drug. The anticancer adjuvant of the present invention can be administered in combination with various types of anticancer drugs or anticancer adjuvants, and when administered in combination, the anticancer drug can be administered at a lower level than the usual dosage of the anticancer drug to show an equivalent level of anticancer treatment effect, so that safer anticancer treatment can be performed.

The route of administration of the anticancer agent may be by any conventional route as long as it reaches the target tissue. The anticancer agents of the present invention may be administered intraperitoneally, intravenously, intramuscularly, subcutaneously, orally, intrapulmonarily, or rectally, depending on the intended use. Furthermore, the anticancer adjuvant may be administered by any device capable of transporting the active substance to the target cell.

The anticancer adjuvant of the present invention may be preferably formulated as an anticancer adjuvant including one or more pharmaceutically acceptable carriers in addition to the active ingredient for administration. Carriers, excipients or diluents that may be included in the anticancer adjuvants of the present invention include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methylhydroxybenzoate, propylhydroxybenzoate, talc, magnesium stearate, and mineral oil.

The anticancer adjuvant of the present invention may be a formulation for oral or parenteral administration, and the description of the formulation is hereby superseded by the description of the pharmaceutical composition.

Hereinafter, the present invention will be described in more detail by way of examples. These embodiments are intended solely to illustrate the invention, and it will be apparent to one of ordinary skill in the art that the scope of the invention is not to be construed as limited by these embodiments.

### Example 1: Preparation of Omeprazole (KN510) Degradation Substance

In the present invention, omeprazole (KN510) was treated with an acid or a base in order to select omeprazole (KN510) degradation substance with superior anticancer effect compared to the conventional omeprazole (KN510).

Specifically, 50 μ [symbol] of 100 mM omeprazole (KN510) was treated with 0, 1, 2, 3, 10, and 20 μ [symbol] of 1 M HCl or 1 M NaOH, respectively, followed by a 1 h reaction at room temperature to prepare omeprazole (KN510) degradation substance.

When omeprazole (KN510) was treated with 1^{˜}20 μ [symbol] of 1 M HCl, omeprazole (KN510) browning substances (acid degradation substances) in the range of pH 2^{˜}6 were prepared, and browning was observed as shown in FIG. 1. Also, when omeprazole (KN510) was treated with 1^{˜}20 μ [symbol] of 1 M NaOH, omeprazole (KN510) basic degradation substances in the range of pH 8^{˜}9 were prepared.

### Example 2: Preparation of Omeprazole (KN510) Browning Substance (Acid Degradation Substance) by Acid Type

In the present invention, to prepare omeprazole (KN510) browning substance (acid degradation substance) according to acid type, 100 μ [symbol] of 100 mM omeprazole (KN510) was treated with 1 M HCl (pH 1^{˜}2), acetic acid (pH 4), and lactic acid (pH 3) at different concentrations (1.5625 μ [symbol] ^{˜}200 μ [symbol] ), and then reacted for 1 hour to prepare omeprazole (KN510) acid degradation substances. As a control, dimethyl sulfoxide (DMSO) at pH 7 was treated.

The pH and absorbance of the omeprazole (KN510) acid degradation substances were then measured using a litmus paper and spectrometer.

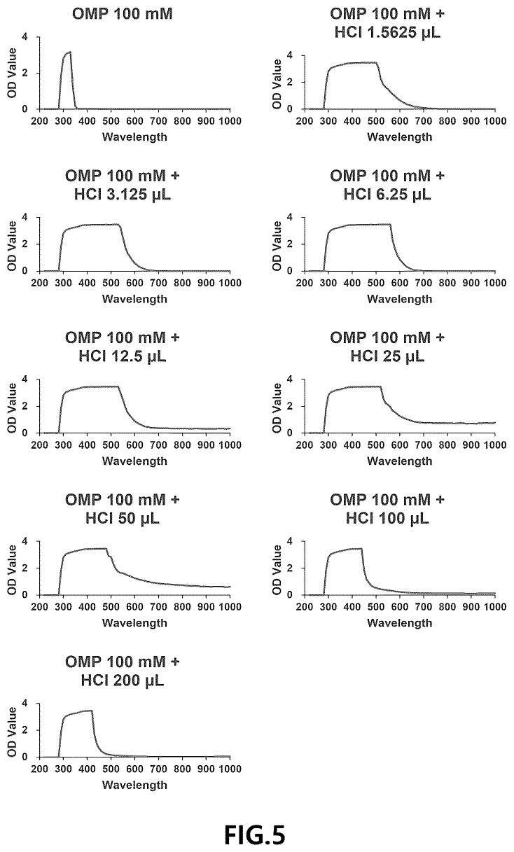

As a result, when omeprazole (KN510) was treated with 1 M HCl, omeprazole (KN510) browning substances (acid degradation substances) with a pH range of 1 to 6 were prepared (FIG. 2), and the absorbance formed a peak of 300 to 500 λm at the pH conditions (FIG. 5).

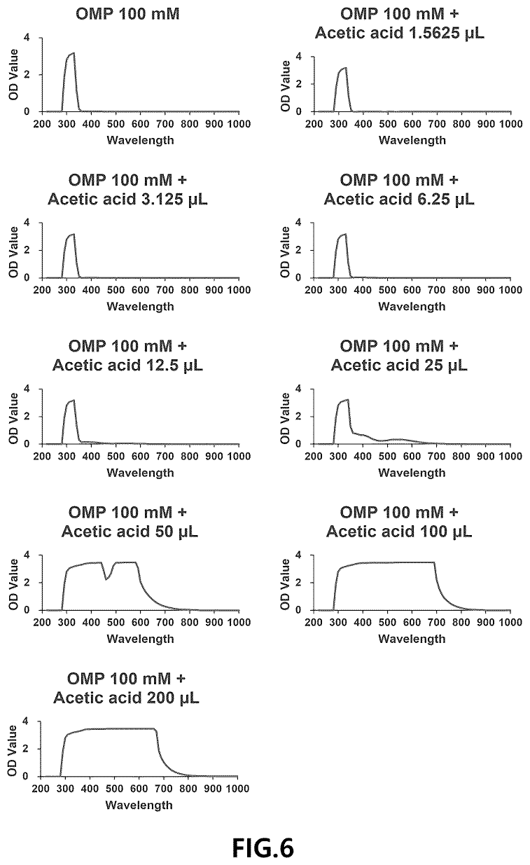

When omeprazole (KN510) was treated with acetic acid, omeprazole (KN510) browning substances (acid degradation substances) with a pH ranging from 3 to 6 were prepared (FIG. 3), and the absorbance formed a peak of 300 to 700 λm at the pH conditions (FIG. 6).

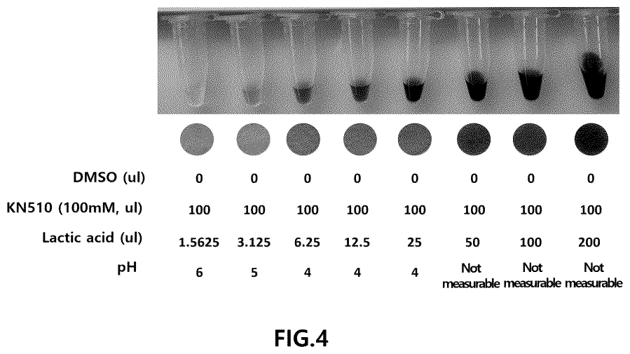

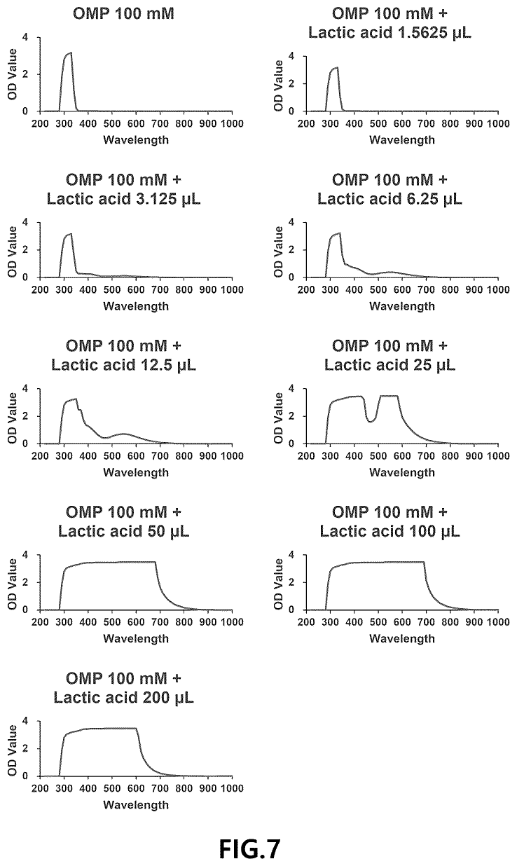

When omeprazole (KN510) was treated with lactic acid, omeprazole (KN510) browning substances (acid degradation substances) with pH ranging from 3 to 6 were prepared (FIG. 4), and the absorbance formed a peak of 300 to 500 λm at the pH conditions (FIG. 7).

### Example 3: Identification of Anticancer Activity of Omeprazole (KN510) Browning Substances

In order to confirm the anticancer activity of the omeprazole (KN510) degradation substances (acid degradation substances) and base degradation substances prepared in Example 1 above, the cancer cell killing effect of each of the omeprazole (KN510) degradation substances was determined.

The extent of cancer cell death was measured by sulforhodamine B colorimetric assay (SRB assay), which was performed as follows: A pancreatic cancer (MIA PaCa2) cell line (100 μ [symbol] ) was inoculated into 96-well cell culture plates at a density ranging from 7,500 to 10,000 cells/well depending on the doubling time. Each of the omeprazole (KN510) degradation substances prepared in Example 1 above was treated at a concentration of 0.1% and incubated in a CO₂ incubator for 48 hours, after which the assay was terminated by adding cold TCA. Cells were fixed intact by gently adding 50 μl of cold 50% (w/v) TCA (final concentration: 10% TCA) and incubated at 4° C. for 60 min. The supernatant was discarded, and the plate was washed five times with distilled water and then air dried. A solution (100 μl) of 0.4% (w/v) sulforhodamine B in 1% acetic acid was added to each well, and the plate was left at room temperature for 10 min. After staining, unbound dye was removed by washing five times with 1% acetic acid, and the plate was air-dried. The bound dye was then solubilized with 10 mM trizma base, and the absorbance was recorded at 515 nm using an automated plate reader. All experiments were performed in triplicate, and data were expressed as mean±standard deviation values.

TABLE 1
Cancer cell growth rate following treatment
with omeprazole (KN510) degradation substance

| KN510 + 1M HCl | | | | | KN510 + 1M NaOH | | | | |
|---|---|---|---|---|---|---|---|---|---|
| | | | aver- | | | | | aver- | |
| | | pH | age(%) | SD | | | pH | age(%) | SD |
| control | | pH 8 | 100.00 | 7.09 | control | | pH 8 | 100.00 | 3.68 |
| 0 | μ [symbol] | pH 8 | 68.15 | 8.37 | 0 | μ [symbol] | pH 8 | 67.18 | 2.40 |
| 1 | μ [symbol] | pH 6 | 61.95 | 3.22 | 1 | μ [symbol] | pH 8 | 64.85 | 6.56 |
| 3 | μ [symbol] | pH 3 | 30.48 | 13.85 | 3 | μ [symbol] | pH 8 | 64.21 | 4.09 |
| 5 | μ [symbol] | pH 3 | −0.81 | 7.59 | 5 | μ [symbol] | pH 8 | 73.66 | 4.25 |
| 10 | μ [symbol] | pH 2 | 5.73 | 9.11 | 10 | μ [symbol] | pH 9 | 68.32 | 2.39 |
| 20 | μ [symbol] | pH 2 | 34.18 | 12.55 | 20 | μ [symbol] | pH 9 | 69.05 | 0.46 |

As a result, as shown in FIG. 8 and Table 1, it was observed that the conventional omeprazole (KN510) and the omeprazole (KN510) basic degradation substances exhibited similar levels of cancer cell growth, but when the cancer cells were treated with omeprazole (KN510) browning substances (acid degradation substances), especially omeprazole (KN510) browning substances (acid degradation substances) in the pH range of 2 to 3, the cancer cell growth was significantly inhibited.

TABLE 2
Cancer cell killing rate (%) by treatment with
omeprazole (KN510) degradation substance

| KN510 + 1M HCl | | | KN510 + 1M NaOH | | |
|---|---|---|---|---|---|
| control | pH 8 | 0.00 | control | pH 8 | 0.00 |
| 0 μ [symbol] | pH 8 | 31.85 | 0 μ [symbol] | pH 8 | 32.82 |
| 1 μ [symbol] | pH 6 | 38.05 | 1 μ [symbol] | pH 8 | 35.15 |
| 3 μ [symbol] | pH 3 | 69.52 | 3 μ [symbol] | pH 8 | 35.79 |
| 5 μ [symbol] | pH 3 | 100.81 | 5 μ [symbol] | pH 8 | 26.34 |
| 10 μ [symbol] | pH 2 | 94.27 | 10 μ [symbol] | pH 9 | 31.68 |
| 20 μ [symbol] | pH 2 | 65.82 | 20 μ [symbol] | pH 9 | 30.95 |

Table 2 shows the cancer cell killing rate (%) compared to the control group using the values in Table 1, and it is confirmed that the conventional omeprazole (KN510) has a cancer cell killing rate of about 32%, while the omeprazole (KN510) browning substance (acid degradation substance) in the pH range of 2^{˜}3 has a cancer cell killing rate of 60%^{˜}100%. It was confirmed that the omeprazole (KN510) browning substance (acid degradation substance) of the present invention has a superior anticancer effect compared to the conventional omeprazole (KN510), and in particular, the present browning substance prepared by treating 50 μ [symbol] of 100 mM omeprazole (KN510) with 5 μ [symbol] of 1 M HCl was found to have the most excellent anticancer effect compared to other browning substances, with a 100% cancer cell killing rate.

### INDUSTRIAL AVAILABILITY

In the present invention, omeprazole browning substance (acid degradation substance) was prepared by treating conventional omeprazole (KN510) with acid, and it was confirmed that the anticancer effect of the present omeprazole browning substance (acid degradation substance) was significantly increased compared to conventional omeprazole. Therefore, the omeprazole (KN510) browning substance (acid degradation substance) of the present invention can be utilized as a composition for preventing or treating cancer, or as an anticancer adjuvant.

1. A pharmaceutical composition for the prevention or treatment of cancer, comprising omeprazole browning substance (acid degradation substance) or a pharmaceutically acceptable salt thereof as an active ingredient.

2. The pharmaceutical composition for the prevention or treatment of cancer of claim 1, wherein the omeprazole browning substance (acid degradation substance) is an acid degradation substance with a pH in the range of 1 to 6.

3. The pharmaceutical composition for the prevention or treatment of cancer of claim 1, wherein the omeprazole browning substance (acid degradation substance) is prepared by treating omeprazole with an acid or acidic solution having a pH ranging from 1 to 6.

4. The pharmaceutical composition for the prevention or treatment of cancer of claim 3, wherein the acid or acidic solution having a pH in the range of 1 to 6 is hydrogen chloride (HCl), acetic acid, or lactic acid.

5. The pharmaceutical composition for the prevention or treatment of cancer of claim 1, wherein the cancer is one or more cancers selected from the group consisting of colorectal cancer, lung cancer, gastric cancer, breast cancer, melanoma, prostate cancer, ovarian cancer, kidney cancer, pancreatic cancer, glioblastoma, and liver cancer.

6. The pharmaceutical composition for the prevention or treatment of cancer of claim 1, wherein the composition further comprises an additional anticancer agent, and the anticancer agent is selected from the group consisting of one or more metabolic inhibitors selected from the group consisting of Irinotecan, fluorouracil, 5-FU), Paclitaxel, Gemcitabine, Cisplatin, Vermurafenib and pharmaceutically acceptable salts thereof; and/or one or more tumor immunosuppressants selected from the group consisting of Pembrolizumab, Nivolumab, Atezolizumab, Ipilimumab, and Durvalumab.

7. An anticancer adjuvant comprising omeprazole browning substance (acid degradation substance) or a pharmaceutically acceptable salt thereof as an active ingredient.

8. The anticancer adjuvant of claim 7, wherein the omeprazole browning substance (acid degradation substance) is an acid degradation substance with a pH in the range of 1 to 6.

9. The anticancer adjuvant of claim 7, wherein the omeprazole browning substance (acid degradation substance) is prepared by treating omeprazole with an acid or acidic solution having a pH ranging from 1 to 6.

10. The anticancer adjuvant of claim 7, wherein the acid or acidic solution having a pH in the range of 1 to 6 is hydrogen chloride (HCl), acetic acid, or lactic acid.

11. The anticancer adjuvant of claim 7, wherein the cancer is one or more cancers selected from the group consisting of colorectal cancer, lung cancer, gastric cancer, breast cancer, melanoma, prostate cancer, ovarian cancer, kidney cancer, pancreatic cancer, glioblastoma, and liver cancer.

12. The anticancer adjuvant of claim 7, wherein the anticancer adjuvant further comprises an additional anticancer agent, and the anticancer agent is selected from the group consisting of one or more metabolic inhibitors selected from the group consisting of Irinotecan, fluorouracil, 5-FU), Paclitaxel, Gemcitabine, Cisplatin, Vermurafenib and pharmaceutically acceptable salts thereof; and/or one or more tumor immunosuppressants selected from the group consisting of Pembrolizumab, Nivolumab, Atezolizumab, Ipilimumab, and Durvalumab.

13. A method for preventing or treating cancer, comprising administering omeprazole browning substance (acid degradation substance) or a pharmaceutically acceptable salt thereof as an active ingredient to a subject in need thereof.
